(19) United States
(12) Patent Application Publication
(10) Pub. No.: US 2010/0297698 A1
(43) Pub. Date: Nov. 25, 2010

(54) SYSTEM AND METHOD FOR PRODUCING SYNTHETIC MICROORGANISMS CAPABLE OF TRANSLATING PROTEINS CONTAINING NON-STANDARD AMINO ACIDS

(76) Inventors: Radha Krishnakumar, Gaithersburg, MD (US); John I. Glass, Germantown, MD (US); Charles E. Merryman, Sykesville, MD (US)

Correspondence Address: Synthetic Genomics c/o MoFo, 12531 High Bluff Drive, Suite 100, San Diego, CA, 92130, US

(21) Appl. No.: 12/681,887

(22) PCT Filed: Oct. 8, 2008
(86) PCT No.: PCT/US08/79229

§ 371 Date: Jul. 9, 2010

(30) Foreign Application Priority Data
Oct. 8, 2007 (US) 60978379

(51) Int. Cl. C12P 21/00; C12N 1/21; C12N 15/63

(52) U.S. Cl. 435/69.1; 435/252.3; 435/471

(57) ABSTRACT

The disclosed invention relates to the generation of host cells containing rare codons and/or absent tRNAs, and the use of orthogonal tRNA systems that can insert a non-standard amino acid into a growing peptide chain. This invention combined with the capacity to synthesize whole genomes has important implications in synthetic biology, as it allows the rewriting of the genetic code of existing or newly designed organisms.

### CROSS-REFERENCE TO RELATED APPLICATIONS

This application claims priority from U.S. provisional application 60/978,379 filed Oct. 8, 2007. The contents of this document are incorporated herein by reference.

### TECHNICAL FIELD

The present invention relates generally to molecular biology, and more particularly to method for using synthetic organisms capable of translating proteins containing non-standard ammo acids to produce custom peptides and proteins.

### BACKGROUND ART

Proteins expressed from the naturally occurring genetic code are generally composed of 20 naturally occurring amino acids. Some microbes utilize the amino acids selenocysteine and/or pyrrolysine at a low frequency in their peptides. A multitude of new peptides and proteins can be produced by incorporating one or more new amino acids, different from these 20, into a peptide or protein during its synthesis.

Currently available methods for inserting amino acid analogs into proteins include: alteration of a cell's protein machinery and using orthogonal tRNA-synthetase systems to suppress nonsense codons; expansion of the genetic code by the inclusion of four-base and five-base codon; non-enzymatic in vitro aminoacylation of tRNA with amino acids that are not among the 20 amino acids that comprise most proteins in life on earth (“non-standard” amino acids, or NSAAs); and modification of the cell's biosynthetic pathways to include the capacity to synthesize and accept NSAAs.

A tRNA synthetase is an enzyme that charges a tRNA with an amino acid, which the tRNA will incorporate into an amino acid chain that is being synthesized during the translation of mRNA into a peptide or protein. In an orthogonal tRNA-synthetase pair, the tRNA and the synthetase react with each other, but the tRNA is not a substrate for any other synthetase that is available in the cell. Similarly, the synthetase acts on the tRNA as a substrate, but not on any other tRNA that is available in the cell. Current methods to synthesize peptides containing NSAAs use suppressor tRNA that recognize the rare amber or ochre codons and insert NSAAs at the site of the stop codons. By the artificial insertion of stop codons into the reading frames of the gene of interest, new peptides and proteins are synthesized that contain NSAAs. Using this method, a maximum of two or three NSAAs can be inserted upon reading one of the stop codons. A limitation of this method is its dependence on the availability of rare, noncoding triplet codons.

To overcome this, some four-base and five-base codons have been generated for use in frameshift suppression. However these systems do not indicate any potential for industrial applications.

Transfer RNAs may be chemically acylated in vitro and imported into cells for site-specific insertion of NSAAs. This method has been used to acylate E. coli suppressor tRNAs with amino acid analogs, and then transfect human cell lines with the acylated tRNAs. The same tRNA molecule can be modified to read more than one codon, and the tRNAs can be acylated with different amino acid analogs, thereby allowing multiple insertions within the same protein. Also, this method abrogates the requirement of plasmid constructions and transformations, since the acylated tRNAs are taken up directly from the growth medium into the cell. However, the tRNA must not be recognized by any endogenous synthetase of the host organism.

A set of tRNAs derived from one single tRNA molecule and mutated at the anticodon region may be used to suppress different codons, but the tRNAs are charged by only one (non-endogenous) synthetase. This method would abolish the requirement of building orthogonal tRNA-synthetase pairs, and allow multiple insertions of amino acid analogs within the same protein. Yet another in vitro acylation method is the use of a bifunctional tRNA that can translate synthetic mRNAs with misacylated tRNAs. This method could be extended for insertion of amino acid analogs in response to sense codons in vitro.

Unassigned codons have been used in the Gram-positive bacterium Micrococcus luteus. In this case, an in vitro translation system was developed for the insertion of NSAAs using synthetically acylated tRNAs that read the unused codons (AGA and AUA) of M. luteus.

There are limitations to the currently available methods for synthesizing proteins containing non-standard amino acids. Only a limited number (a maximum of two or three) of non-standard amino acids has been inserted thus far. Also, these methods require several genetic manipulations of the host organism and of the protein to be expressed in the host organism. None of the currently available methods is efficient for in vivo production of proteins containing NSAAs.

### SUMMARY OF THE INVENTION

One embodiment of the invention relates to a method expressing a protein containing one or more non-standard amino acids, comprising: providing a host organism with a genome, wherein the genome contains a gene for the protein, wherein the gene comprises one or more target codons; providing an orthogonal tRNA system comprising a non-standard tRNA, a non-standard aminoacyl-tRNA synthetase (NSAARS), and a non-standard amino acid, wherein the NSAARS charges the NStRNA with the non-standard amino acid; culturing the organism under conditions where the protein is expressed and the protein contains the non-standard amino acid.

Another embodiment of the invention relates to a method of engineering a microorganism dependent on a non-standard amino acid for survival, comprising: providing a host organism with a genome, wherein the genome contains a gene for an essential protein, wherein the gene comprises one or more target codons; providing an orthogonal tRNA system comprising a non-standard tRNA, a non-standard aminoacyl-tRNA synthetase (NSAARS), and a non-standard amino acid, wherein the NSAARS charges the NStRNA with the non-standard amino acid; culturing the organism under conditions where the protein is expressed and the protein contains the non-standard amino acid and wherein failure to express the essential protein results in the death of the host organism.

In aspects of the disclosed invention the host organism is a eukaryote or a prokaryote. For example, the host organism can be a bacteria, such as M. capricolum. In another aspect of the invention, the non-standard tRNA (NStRNA) is obtained from a yeast, an Archeabacteria, or a fungus and the non-standard aminoacyl tRNA synthetase (NSAARS) is obtained from yeast. The NSAARS need not possess an amino acid proofreading function. Particular examples of NSAARSs contemplated for use with the disclosed invention include NSAARSs charges a tRNA molecule with an amino acid selected from the group consisting of asparagine, aspartic acid, cysteine, histidine, lysine, and tryptophan. In a particular embodiment, the NSAARS charges a tRNA molecule with an arginine amino acid. The invention contemplates the use of NStRNAs from yeast and NSAARSs from yeast. For example, the NSAARS can be from Nanoarchaeum equitans or Schizosaccharomyces pombe.

### BRIEF DESCRIPTION OF THE DRAWINGS

FIG. 1 shows a table with 30 non-standard amino acids.

FIG. 2 shows a table with the genetic code and codon usage and frequency for M. capricolum.

FIG. 3 shows the graphical representation to two tRNA molecules with anticodons.

FIG. 4 shows a gel with the results of a tRNA charging experiment.

FIG. 5 shows a gel with the results of a tRNA charging experiment with tRNA^{CCG} of N. equitans and M. capricolum ArgS.

FIG. 6 shows a gel with the results of a tRNA charging experiment where the ArgS of N. equitans charged the tRNA Arg of M. capricolum.

FIG. 7 show a gel with the results of a tRNA charging experiment where the k_{obs} of yeast ArgS was approximately 27-fold less than k_{obs} of M. capricolum ArgS.

FIG. 8 shows a gel with the results of a tRNA charging experiment where the k_{obs} of yeast ArgS was 13-fold less than the k_{obs} of M. capricolum ArgS.

FIG. 9 shows a gel of a tRNA charging experiment demonstrating that yeast ArgS does not charge yeast tRNA^{CCG}.

FIG. 10 shows a gel of a tRNA charging experiment demonstrating that N. equitans tRNA^{CCG} was not aminoacylated by M. capricolum, but was charged by the yeast ArgS.

FIG. 11 shows a gel of a tRNA charging experiment demonstrating that the tRNA^{CCG} of N. crassa was aminoacylated by yeast ArgS, but not by M. capricolum.

FIG. 12 shows a gel examining the capacity of tRNA_{Arg} from M. capricolum to be charged with various arginine analogs, 1. L-arginine, 2. L-canavanine, 3. NG-monomethyl-L-arg, 4. NG-hydroxyl-L-arg, 5. Vinyl-L-NIO.

Legend for figures: ne-N. equitans; nc-N. crassa; mc-M. capricolum; and y-yeast or S. cerevisiae.

### DETAILED DESCRIPTION OF THE INVENTION

Embodiments of the present invention provide a system and method for the synthesis of an organism that may code for amino acids beyond the traditional 20, and which may thus produce peptides and proteins customized by the incorporation of these non-standard amino acids (NSAAs). The customized peptides and proteins may be designed to have new or improved physical properties and functions. By involving the synthesis of a microbial genome that would lack one or more codons in all genes except the genes that encode peptides intended to contain NSAAs, these embodiments begin upstream of the methods currently practiced to obtain custom proteins. The tailored microorganisms of these embodiments may be able to produce custom proteins in quantities (such as gram quantities) several orders of magnitude greater per time spent preparing the proteins than can be achieved by any currently available means, so that it may become cost-effective to produce infinitely more custom peptides and proteins according to the present invention than could be made otherwise. Some embodiments also allow one skilled in the art to perform screens and selections that identify mutant organisms that have evolved to gain useful properties due to the inclusion of the NSAAs. Thus, these cost-effective embodiments vastly broaden the field of custom peptides and proteins that can be obtained for biochemical, biomedical, biotechnical and materials research and applications.

The described invention manipulates sense and nonsense codons as targets for incorporating non-standard amino acids into growing peptide, which are translated from messenger RNA (mRNA) produced by a host cell. The methods described herein exploit the role transfer RNA (tRNA) molecules play in protein production. tRNA molecules are true adaptor molecules in that it is the anticodon alone that determines which amino acids charged to particular tRNAs are incorporated into a growing peptide. Thus the interaction of particular tRNA molecules and their anticodons with particular codons can be manipulated to incorporate amino acids other than the 20 normally found in proteins into in vivo expressed proteins. The methods described herein provide guidance to one of ordinary skill in the art to identify suitable nonsense, or rare codons that can be targeted for use in proteins as targets for non-standard amino acids. The described methods also provide direction as to how appropriate adaptors can be charged with non-standard amino acids by selection of the appropriate aminoacyl tRNA synthetases and non-standard amino acids. Uses for the described methods are also provided.

Selection of A Target Codon

A preliminary step to selecting a target codon that can be exploited to insert non-standard amino acids (NSAAs) into in vivo produced proteins and peptides involves selecting a suitable host organism. Sense suppression in a host involves selecting one or more under utilized or rare codons that can be used as targets for tRNAs charged with non-standard amino acids. Nonsense suppression involves the exploitation of a nonsense or stop codon as the target for the tRNA charged with an NSAA.

Preferably, the host organism will possess a genome comprising one or more target codons. The target codons comprise the sites where the NSAAs are incorporated into the translated gene products. In a preferred embodiment the host cell comprises a genome which codes for one or more nonsense codon, one or more rare codons, or a combination of both. A “nonsense codon” is a codon that does not possess a cognate transfer RNA (tRNA). Translation will usually cease after a ribosome encounters a nonsense codon because of the lack of a cognate tRNA to supply the next amino acid for the growing peptide chain. A “rare codon” is a codon that is used rarely by the host organism in the proteins it expresses. Typically, a rare codon is found from 0.00 to 0.50%, 0.00 to 0.25%, 0.00 to 0.10%, 0.00 to 0.05%, or 0.00 to 0.01% of all codons in a host genome.

Inserting novel amino acids into the genetic code of a host organism can be achieved by generating an organism that is free of one or more sense codons, for example, by deleting the target codon to produce a “codon-free” organism. For most organisms, this requires numerous chromosomal alterations. To narrow the possibility of generating lethal mutations, all instances of the target codon is first be replaced with an alternative codon that specifies the same amino acid or a similar amino acid. Next, the tRNA or tRNAs responsible for reading the target codon are removed from the host genome. Finally, the target codon and an appropriate tRNA are then provided to the host organism in such a way as to specify a novel, non-standard, or unnatural amino acid. Such manipulations make it possible to expand the genetic code well beyond the traditional 20 amino acids.

A tailored host genome could be constructed by inserting modified sections of the genome until the “codon-free” state was achieved. Alternatively, an entirely synthetic genome can be constructed (Gibson et al. Science (2008) 319:1215-20) with the desired codon codes, where the synthetic genome is then used to produce host proteins. The genetic sequence of a synthetic microorganism can be designed in its entirety, thus allowing for one of ordinary skill in the art to direct in the incorporation of NSAAs into one or more genes of interest. Nonetheless, limiting the number of changes required to generate a, synthetic microorganism's chromosomal sequence from a naturally occurring nucleic acid sequence simplifies the construction of an organism that is free of a specific codon, and thereby simplifies the subsequent expansion of the genetic code to include NSAAs. In some embodiments, a naturally occurring nucleic acid sequence is used as a starting material for creating a synthetic genome and/or a synthetic organism that can synthesize proteins having one or more amino acids that is not one of the standard 20 amino acids.

Host organisms with genomes that contain rare codons, whether sense or nonsense, require only a small number of alterations to be made free of a specific codon. Some embodiments of the invention-comprise “codon-free” organisms made by limited alteration of a naturally occurring genome having one or more underrepresented codons. Exemplary embodiments comprise the re-introduction of the deleted, absent, rare or otherwise underrepresented codon into one or more specific regions of the genome and/or into one or more foreign genes, the expression of which are desired in the codon-free organism, as well as the alteration of the amino acid specified by the codon. This may be achieved by introducing one (or more) artificially synthesized tRNA and its (or their) cognate amino-acyl tRNA synthetase capable of recognizing the reintroduced codon and inserting NSAAs or amino acid analogs. The resulting orthogonal system allows the insertion of NSAAs in response to any rarely used or unassigned codons. By using sense codon suppression, any dependence on the availability of non-coding triplets is abolished (this dependence is one of the major limitations of currently available systems for inserting NSAAs). A “codon-free” organism could be made via traditional molecular techniques such as homologous recombination and site-directed mutagenesis.

Depending on the codon usage in the host, any organism that can be cultured can be used with the methods described herein. For example, both eucaryotic and prokaryotic organisms can be used with the present methods. In one embodiment, the naturally occurring genome of an organism that rarely uses a specific codon, such as Mycoplasma capricolum subspecies capricolum strain California kid, may be used. This strain of M. capricolum has only 29 tRNA species, and only two tRNAs exist to translate the six potential arginine codons. For CGN-type arginine codons there is only one tRNA, and it has an ICG anticodon. (The other arginine tRNA present in M. capricolum is used to decode AGR-type arginine codons, and thus it is not involved in reading the CGN codon set.) Mycoplasma mycoides subspecies mycoides small colony phenotype is another example of a type of Mycoplasma hat has only 29 amino acids and does not encode the tRNA^{CCG}, just like M. capricolum.

According to wobble rules, tRNA ICG may read CGU, CGC and CGA codons, but not CGG codons. The M. capricolum genome reveals six CGG codons in the open reading frames of putative proteins. Of these, two of the genes are non-essential, and none of the six codons is conserved across related species of Mycoplasma. Translation of the CGG codon is inefficient both in vivo and in vitro, and is one of the reasons for poor translation of certain reporter genes such as beta-galactosidase. Because of the rarity of the CGG codons, their replacement by any combination of CGU, CGC, CGA, AGA, or AGG arginine codons requires only six alterations of the M. capricolum genome.

Using the naturally occurring genome of an organism with rare instances of a specific codon further simplifies the subsequent expansion of the genetic code to include NSAAs. Because of the absence of an existing tRNA able efficiently to decode CGG in M. capricolum, and because the genes containing CGG codons are non-essential or are not conserved, it is unnecessary to change the existing CGG codons present in the M. capricolum chromosome. By engineering and supplying a tRNA capable of reading and inserting non-standard amino acids in response to CGG-sense codons, any miscoding that normally occurs at the six instances of CGG is likely to be insignificant in comparison to decoding by the alternative endogenous tRNA that reads through these codons. Thus, the primary chromosomal, extrachromosomal or physical alteration required in some embodiments is to supply a tRNA capable of efficiently decoding a rare or underutilized codon in M. capricolum, to generate an organism that utilizes a NSAA.

One of skill in the art will readily appreciate the applicability of this technology to other organisms that contain one or more rarely used codons, regardless of the presence or absence of the tRNA reading such a codon. Generating a microorganism that may utilize one or more non-standard amino acids as a part of its normal metabolism may be achieved by various methods, including but not limited to: tRNA deletion and an intermediate stage requiring miscoding by alternative tRNAs; simultaneous deletion of the existing tRNA and addition of an alternative tRNA; and direct swapping of the existing tRNA with a system capable, of inserting one or more NSAAs. Various embodiments include altering the tRNA decoding specificity of organisms that are naturally limited but not completely free of one or more sense codons. Some embodiments involve the re-introduction of the limited codon and the alteration of the amino acid it specifies.

Once an organism with a genome devoid of the CGG codon (or any other rare codon) is obtained, the codon may be reintroduced into specific genes by several methods. Methods such as chemical mutagenesis to create base analogs and site-directed mutagenesis can be combined with homologous recombination to insert the rare codon into specific regions of the genome. Some embodiments include the introduction of rare or unassigned codons into synthetic genomes, thus leading to the de novo synthesis of an organism with the capability of utilizing NSAAs in peptide or protein synthesis.

Charging the tRNA with a NSAA

Once the desired codon has been introduced or reintroduced into the organism, methods for reading the codon and inserting NSAAs or amino acid analogs in response are applied. One such method is the creation of an orthogonal tRNA system, which comprises a non-standard tRNA (NStRNA), a non-standard amino-acyl tRNA-synthetase (NSAARS), and a non-standard amino acid, where the resulting charged NStRNA specifically reads the target codon, and in response inserts a NSAA. This approach exploits the isolation and/or synthesis of a NStRNA species that is not recognized by the tRNA amino-acyl synthetases of the host organism, and a NSAARS that will exclusively acylate the NStRNA. Such orthogonal such orthogonal systems have been developed from tRNAs and synthetases from some archaea for insertion of NSAAs into proteins expressed in eubacteria such Escherichia coli and Bacillus subtilis, as well as in some eukaryotic systems. These orthogonal pairs use tyrosine, lysine, glutamate, leucine or glutamine tRNA-synthetase pairs.

The general methodology involves selecting a tRNA adaptor molecule, an appropriate aminoacyl tRNA synthetase, and non-standard amino acids for incorporation into a gene of interest.

tRNA Adaptor Molecules

The sequences of several hundred transfer RNA (tRNA) molecules from a variety of very different organisms have been sequenced. The data provided shows that tRNA molecules comprise a plurality of conserved or invariant residues with a number of semi-variant bases distributed in various portions of the molecule. The profound understanding of the functional components of tRNA molecules enables one of ordinary skill in the art to construct orthogonal systems which can be introduced into a host cell to incorporate non-standard amino acids into expressed proteins.

Transfer RNA molecules are typically depicted in a cloverleaf format. This presentation highlights the hydrogen bonding present within a tRNA and allows one to highlight particular bases within the molecule that are thought to play a functional role. Moving from 3′ to 5′, relevant portions of a tRNA include the acceptor stem at the 3′ end of the molecule, upon which an amino acid is attached through the catalytic action of a cognate aminoacyl tRNA synthetase, the TΨC loop, a variable loop, the anticodon loop, the D loop which contains the α and β regions, and finally the 5′ end of the acceptor stem. Certain positions of tRNA molecules are conserved, for example the terminal 3′ end of a tRNA molecule has the sequence CCA. The 5′ end of the tRNA molecule consistently contains a 5′-terminal phosphate. Various regions of a tRNA molecule, including the acceptor region, are utilized by aminoacyl tRNA synthetases to discriminate between tRNA adaptor molecules so that only tRNA molecules with the correct anticodon are charged with the correct cognate amino acid.

The 3′ terminal adenosine of the tRNA is charged with an amino acid residue via a covalent bond formed between the amino acid carboxyl group and the terminal phosphate. The aminoacyl tRNA synthetase scrutinizes the union of amino acid and tRNA. Some synthetases must discriminate between amino acid residues that share a high degree of structure, such as isoleucine and valine or phenylalanine and tyrosine. It is likely that it will be more difficult to find non-standard amino acid analogs for these amino acids, since the aminoacyl tRNA synthetases that charge the cognate tRNAs have a proofreading function. In contrast, other amino acids that can be discriminated without proofreading are more likely to be usable as vehicles for incorporated non-standard amino acids into in vivo translated proteins. These include asparagine, aspartic acid, cysteine, histidine, lysine, and tryptophan. Each of the aminoacyl tRNA synthetases that charge tRNA adaptors with these amino acid residues are potential vectors for use with the presently described invention.

A number of methods are available to produce tRNAs charged with a desired NSAA. In one embodiment, after a host cell is selected, different exogenous tRNAs can be tested for their ability to be charged by endogenous host cell aminoacyl tRNA synthetases. Preferably an exogenous tRNA is selected that is not charged by an endogenous enzyme. To complete the orthogonal pair, an exogenous aminoacyl tRNA synthetases is selected that is capable of charging the exogenous tRNA adaptor molecule, preferably with the desired NSAA.

It is possible to alter a tRNA in the anticodon region to read a target codon, which is a rare codon or a nonsense codon. The synthetase specificity for the tRNA or the NSAA can also be altered through a directed evolution method, such that it acylates only the cognate tRNA molecule with NSAAs and not the endogenous tRNAs. In this embodiment, regions of the enzyme that come in contact with the amino acid are mutagenized, and synthetase variants that can acylate the tRNA with NSAAs are selected through several rounds of positive and negative selection. It will be appreciated by one of skill in the art that reading the codon and inserting a desired amino acid may be achieved by other methods and remain within the scope of the various methods provided herein.

Non-Standard Amino Acids

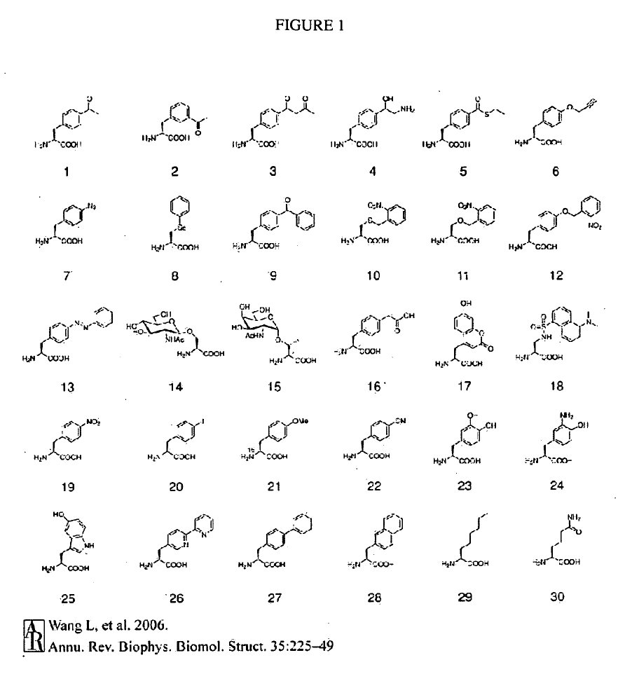

A growing variety of non-standard amino acids can be used with the presently described invention. For examples, β-amino acids (β³ and β²), homo-amino acids, cyclic amino acids, aromatic amino acids, Pro and Pyr derivatives, 3-substituted Alanine derivatives, Glycine derivatives, ring-substituted Phe and Tyr derivatives, linear core amino acids, and diamino acids, which are all available commercially from SIGMA-ALDRICH, are contemplated for use with the presently described methods. A table of exemplary NSAAs that have been incorporated into E. coli proteins is provided in FIG. 1.

Utility

Some embodiments comprise a system and method of inserting NSAAs into proteins by creating an organism whose genome encodes these amino acids. Exemplary, embodiments employ sense codon suppression, such as suppression of the arginine codon CGG in M. capricolum, using this rare sense codon for the insertion of NSAAs, and thereby generating a synthetic organism capable of utilizing NSAAs in peptide and protein synthesis.

Because of the rarity of the CGG codon in Mycoplasma, and especially in the Mycoides cluster of Mycoplasmas, a tRNA molecule may be designed to charge with amino acid analogs such as pyrrolysine, selenocysteine, as well as any other NSAAs or amino acid analogs that are not toxic to the codon-free organism. By using the rare codon suppression technique in Mycoplasma, any protein that can tolerate amino acid analogs in permissive sites can be synthesized without manipulating the peptide and/or host microorganism at the genetic level. Some embodiments of the invention include the creation of a Mycoplasma genome with the capability of translating CGG codons that will allow the use of common reporter systems and selection markers in this organism. This system essentially allows the rewriting of the genetic code of M. capricolum, and of any other eubacterium for which the system is adapted. Thus, some embodiments of the invention involve the alteration of the genetic code of microbial genomes so that artificially synthesized tRNAs may be used to insert NSAAs into peptides and/or proteins expressed by these genomes.

While various embodiments and methods have been described herein, it should be understood that they have been presented by way of example only, and not limitation. Further, the breadth and scope of a preferred embodiment should not be limited by any of the above-described exemplary embodiments.

### EXAMPLE 1

### Selection of a Host

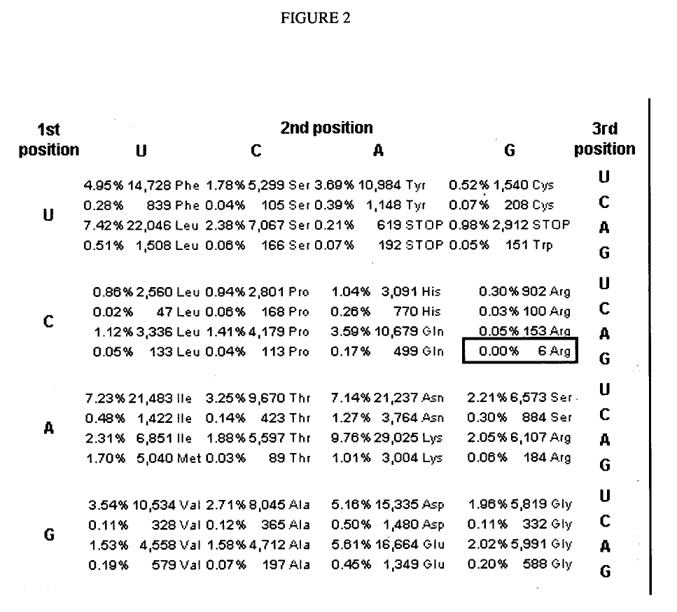

Mycoplasma capricolum was selected as the host organism to capitalize on phenomenon of near absence of a sense codon (CGG) coupled with the absence of its cognate tRNA, to create an orthogonal system that will insert amino acid analogs in response to the CGG codon. M. capricolum, has a rare sense codon CGG that occurs only six times through the entire genome, and more importantly it lacks the tRNA with correct anti-codon (tRNA^{CCG}) to read this codon. (See FIG. 2.)

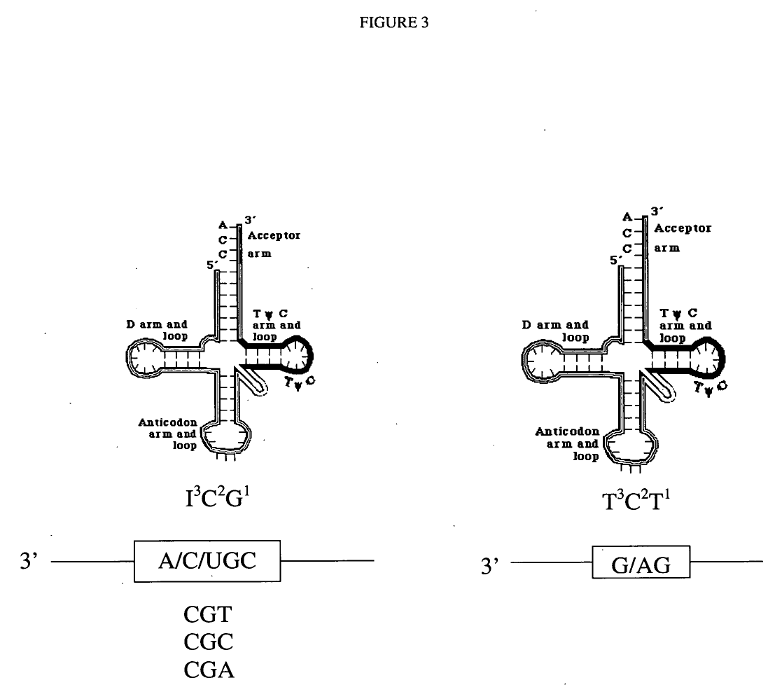

Since Inosine (I), through Watson-Crick or wobble base-pairing can pair with T, C and A, but not G, three of the CGN codons are read by a single tRNA^{ICG}. The codon AGA is read by tRNA^{TCT}. (See Table 1 and FIG. 3.)

TABLE 1

| Isotype | Anticodon count | | | | | | Total |
|---|---|---|---|---|---|---|---|
| Arg | ACG 1 | GCG | CCG | TCG | CCT | TCT 1 | 2 |

There is no tRNA to read the CGG codon. Although none of the six genes that contain the CGG codon appear to be essential, it is clear that there is some read-through of the these codons, possibly by tRNA^{ICG}, as is evident from the weak but consistent expression of foreign reporter genes containing this codon in M. capricolum.

To create such a system three components are selected, 1) tRNA_{Arg} not recognized by endogenous M. capricolum amino-acyl synthetases; 2) Arginyl-tRNA-synthetase (AARS) that cannot aminoacylate endogenous tRNAs; and 3) Analogs of amino acids utilized by the above two components, but not by endogenous translation system.

### EXAMPLE 2

### tRNA^{Arg} Not Recognized by Endogenous M. capricolum ArgS

The enzymatic reaction provided below shows the charging of a tRNA for arginine.

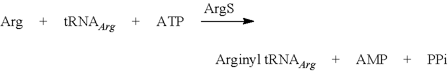

tRNAs and arginyl-tRNA-synthetases are screened from phylogenetically diverse organisms and/or with unique recognition elements to identify suitable tRNAs for use with the disclosed invention. Screening candidates include the eubacterium E. coli, the fungal species—Saccharomyces cerevisiae and Neurospora crassa, and one member of the Archaeal branch, Nanoarchaeum equitans.

In vitro transcription of tRNAs using T7 RNA polymerase is performed. The transcripts are purified from 10% acrylamide gel (7.5 M urea-TBE).

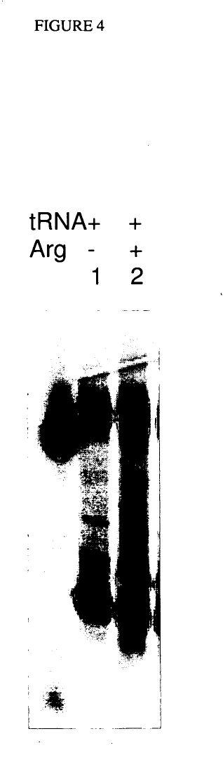

Charging tRNAs is detected using tRNA, ArgS (an arginyl synthetase), and rATP. The mix is incubated at 37 C for 0-15 minutes. The reaction is quenched by adding NaOAc, EDTA, and urea. The recovered material is loaded on a 0.25 mm, 6% acrylamide gel containing 7.5 M urea, 100 mM NaOAc, ph 5.0. The gel is run at 4 C, 500 v, 3000 vhours, and stained with SYBRgold (INVITROGEN). Exemplary results are shown in FIG. 4. Lane 1 shows the results from a control sample with tRNA and no Arg. Lane 2 shows the results from a reaction which contained both the tRNA and Arg. The additional band visible in lane 2 shows the charging of the tRNA.

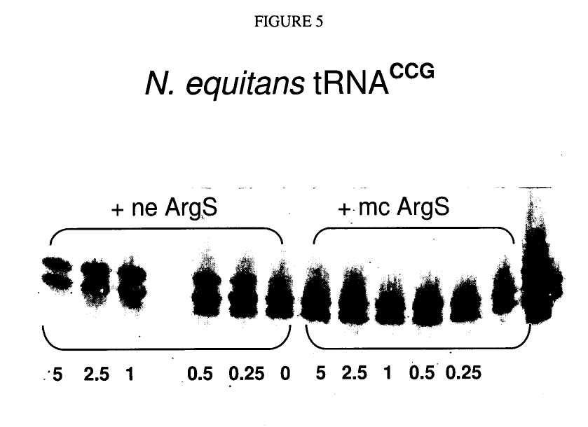

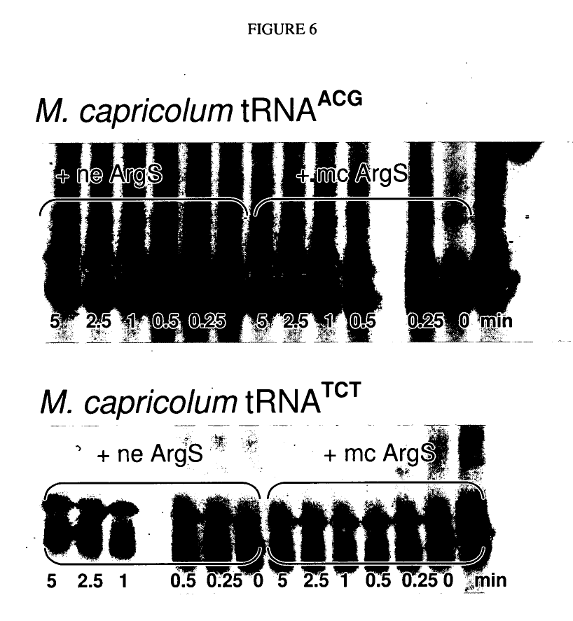

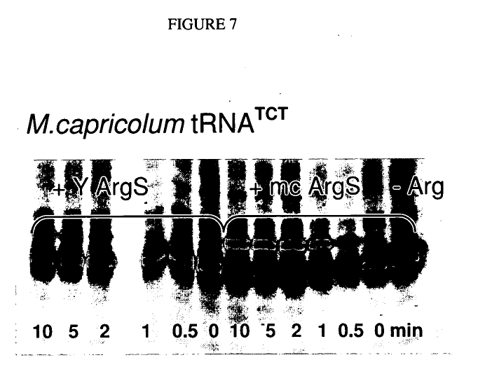

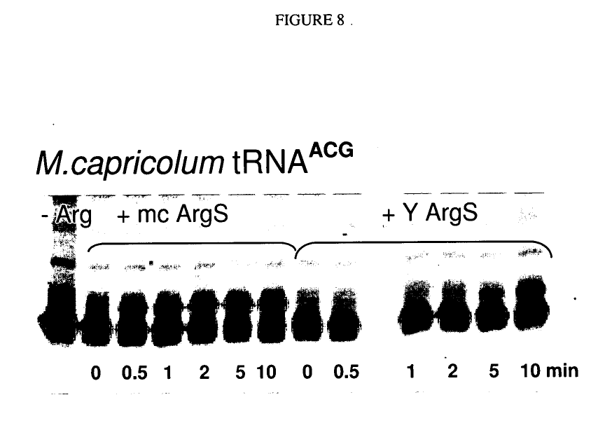

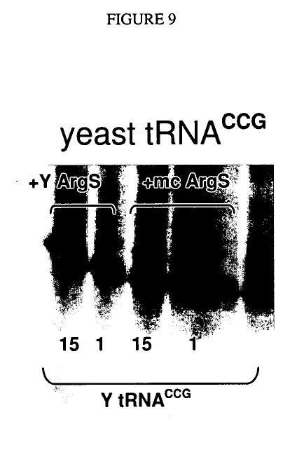

A similar methodology was used to compare the charging of tRNA Arg from N. equitans, N. crassa, E. coli, S. cerevisiae with tRNA_{Arg} of M. capricolum. As shown in FIG. 5, the tRNA^{CCG} of N. equitans was not charged by M. capricolum ArgS. But as shown in FIG. 6, ArgS of N. equitans does charge tRNA Arg of M. capricolum. The results shown in FIGS. 7 and 8 show the interactions. Yeast tRNA^{CCG} was not charged by M. capricolum ArgS. tRNA Arg of M. capricolum was charged by yeast ArgS but at rates much lower than it's own synthetase. Interestingly, yeast ArgS does not charge yeast tRNA^{CCG}. (See FIG. 9.) Yeast tRNA^{CCG} has a modified base (dihydrouridine) at position U20. DHU20 is required for aminoacylation of tRNA^{CCG} by yeast ArgS.

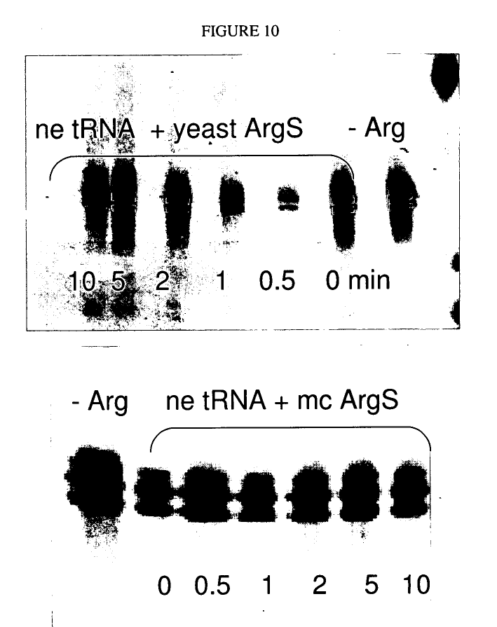

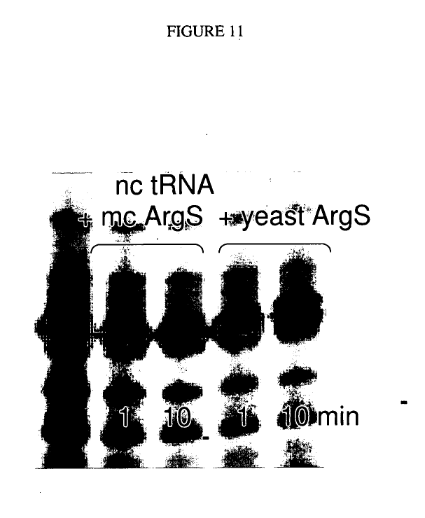

Results using N. equitans tRNA^{CCG} and yeast ArgS indicate these components as being suitable for use in an orthogonal system, since N. equitans tRNA^{CCG} was not aminoacylated by M. capricolum, but was charged by the yeast ArgS. (See FIG. 10.) Similarly, the tRNA^{CCG} of N. crassa was aminoacylated by yeast ArgS, but not by M. capricolum. (See FIG. 11.)

The results presented here demonstrate three potential orthogonal systems that can be used to introduce non-standard amino acids into peptides.

### EXAMPLE 3

### Identification of Analogs of Arginine Not Utilized by M. capricolum Translation System

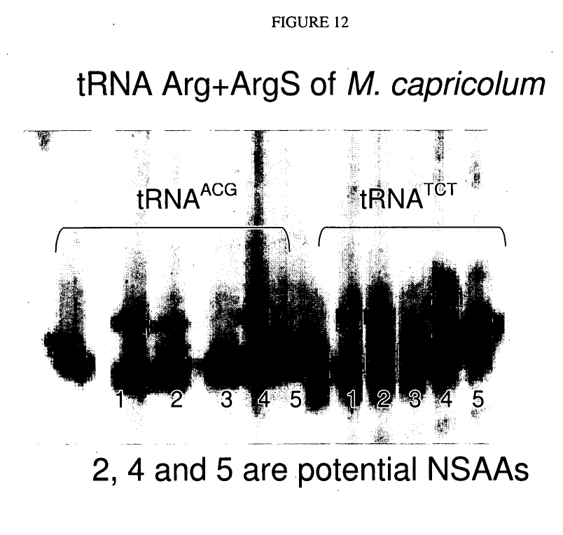

A variety of analogs of arginine (canavanine, NG-monomethyl-L-arginine (L-NMMA), NG-hydroxyl-L-arginine, and vinyl-L-NIO) were examined for use with the current invention. As shown in FIG. 12, canavanine, NG-monomethyl-L-arginine (L-NMMA), and vinyl-L-NIO are likely candidates as arginine analogs because they are not synthesized or used by M. capricolum.

### EXAMPLE 4

### M. capricolum with a Yeast Orthogonal Pair

A host organism M. capricolum is engineered with an orthogonal pair obtained from Saccharomyces cerevisiae where the host cell contains the exogenous tRNA^{CCG}-arginyl synthetase pair. The exogenous orthogonal pair produces a tRNA^{CCG} charged with an non-standard amino acid. This pair does not cross charge with the components of M. capricolum. A protein of interest is encoded by an exogenous vector and is introduced into the engineered M. capricolum. The engineered host organism is cultured under conditions to permit expression of the protein of interest and because of the presence of yeast orthogonal pair, the protein contains the non-standard amino acid.

### EXAMPLE 5

### M. capricolum with an Exogenous Orthogonal Pair

A host organism M. capricolum is engineered with an orthogonal pair where the exogenous arginyl synthetase is obtained from S. cerevisiae and the tRNA^{CCG} a N. equitans. The exogenous orthogonal pair produces a tRNA^{CCG} charged with an non-standard amino acid. This pair does not cross charge with the components of M. capricolum. A protein of interest is encoded by an exogenous vector and is introduced into the engineered M. capricolum. The engineered host organism is cultured under conditions to permit expression of the protein of interest and because of the presence of the exogenous orthogonal pair, the protein contains the non-standard amino acid.

### EXAMPLE 6

### M. capricolum with an Exogenous Orthogonal Pair

A host organism M. capricolum is engineered with an orthogonal pair where the exogenous arginyl synthetase is obtained from S. cerevisiae and the tRNA^{CCG} from N. crassa. The exogenous orthogonal pair produces a tRNA^{CCG} charged with a non-standard amino acid. This pair does not cross charge with the components of M. capricolum. A protein of interest is encoded by an exogenous vector and is introduced into the engineered M. capricolum. The engineered host organism is cultured under conditions to permit expression of the protein of interest and because of the presence of the exogenous orthogonal pair, the protein contains the non-standard amino acid.

### EXAMPLE 7

### Selection System for the Insertion of Arginine Analog

A selection system is used to force M. capricolum into using the orthogonal tRNA^{CCG}/synthetase. A selection system conveying tetracycline resistance using the tetM gene, which confers tetracycline resistance in Mycoplasma, is introduced into a host cell. Multiple CGG codons at the beginning of the open reading frame of tetM slow down the ribosome and prevent full length tetracycline resistance protein from being made unless tRNA^{CCG} are incorporated into the gene product produced from the tetM gene. Cells expressing the mutated tetM gene (with multiple CGGs at the beginning of the open reading frame) from the plasmid with the tetM gene are unable to grow in the presence of tetracycline in the medium. Those cells that utilize the orthogonal tRNA^{CCG}-arginyl synthetase show normal growth.

To show that such an insertion occurred an exogenous test protein protein (β-galactosidase) known to contain multiple CGGs and expressed well in M. capricolum is purified. β-galactosidase is selected because it contains seven CGG codons. This protein is also expressed from the construct that carries the mutated tetM gene with multiple CGG insertions and the yeast orthogonal pair. Immunoprecipitation of the β-galactosidase protein from the wild type strain and the strain carrying the orthogonal pair, followed by mass spec analysis reveals the presence of arginine analogs.

### EXAMPLE 8

### Insertion of Lysine Analogs Through Sense Codon Suppression

Lysine analogs represent an alternative orthogonal pair. This example uses tRNA^{pyl} and pyrrolysyl synthetase (PylRS) from Methanogens. The structure of tRNA^{pyl} is different from all other tRNAs and therefore cannot be charged by any other aminoacyl-synthetase. The anticodon of tRNA_{pyl} is CUA and reads in-frame stop codons UAG, to insert pyrrolysine. The anticodon is not an identity element, so when this is changed to CCG, it will still be recognized by the pyrrolysyl synthetase and insert lysine analogs structurally similar to pyrrolysine at positions of the CGG codon.

This is the system is particularly well suited for the construction of a synthetic cell incapable of growing in the absence of a specific amino acid analog. The capacity to create such NSAA auxotrophs would enable creation of synthetic microorganisms that could not survive in the absence of human application of the necessary NSAA to the growth medium, host organism, or environment. Imagine treating a patient with a synthetic microorganism that had both immediate therapeutic and long term pathogenic capacity. Coincident with the treatment the patient would be given the NSAA needed for the organism for growth. Once the therapy was complete application of the NSAA would be ceased and the synthetic organism would die. An essential protein of in a synthetic Mycoplasma host cell is selected or altered to provide for the presence of multiple CGG codons. Because the protein is essential for growth, the organism can only survive if it carries the tRNA^{pyl} and pyrrolysyl synthetase and when grown in the presence of a lysine analog.

1-29. (canceled)

30. A host organism comprising a synthetic genome, wherein the organism comprises an orthogonal tRNA system comprising a non-standard tRNA and a non-standard aminoacyl-tRNA synthetase (NSAARS), and further wherein the synthetic genome is designed in its entirety and contains at least one gene of interest comprising at least one target codon for the incorporation of at least one non-standard amino acid into an expression product of the at least one gene of interest.

31. A host organism according to claim 30, wherein the host organism is a eukaryote or a prokaryote.

32. A host organism according to claim 31, wherein the prokaryote is a bacteria.

33. A host organism according to claim 30, wherein the non-standard tRNA is obtained from a yeast, an Archeabacteria, or a fungus.

34. A host organism according to claim 30, wherein the NSAARS is obtained from yeast.

35. A host organism according to claim 30, wherein the at least one target codon in the at least one gene of interest replaces at least one underutilized or rare sense codon in the host organism.

36. A host organism according to claim 30, wherein the at least one target codon in the at least one gene of interest replaces at least one nonsense or stop codon in the host organism.

37. A host organism according to claim 30, wherein the at least one target codon in the at least one gene of interest is added to the existing set of standard codons in the host organism.

38. A host organism according to claim 30, wherein the host synthetic genome is designed to be free of one or more sense codons.

39. A host organism according to claim 30, wherein the at least one non-standard amino acid is selected from the group consisting of an arginine analog, a lysine analog, and a cysteine analog.

40. A host organism according to claim 30, wherein the host organism is a non-standard amino acid auxotroph wherein the at least one gene of interest is a gene encoding an essential protein and the host organism is dependent on the at least one non-standard amino acid for survival.

41. A method of regulating the growth of a host organism according to claim 40, said method comprising culturing the host organism in the presence or absence of the non-standard amino acid under conditions wherein (i) the essential protein is expressed in the presence of the at least one non-standard amino acid and (ii) the essential protein is not expressed in the absence of the at least one non-standard amino acid resulting in death of the host organism.

42. A method of expressing a protein containing at least one non-standard amino acid, said method comprising culturing a host organism according to claim 30 with the at least one non-standard amino acid under conditions wherein the protein is expressed and the NSAARS charges the non-standard tRNA with the at least one non-standard amino acid to provide the protein containing the at least one non-standard amino acid.

43. A method of engineering an organism dependent on a non-standard amino acid for survival, said method comprising :
(i) modifying the genome of a host organism to provide the host organism with a genome comprising at least one gene encoding an essential protein, wherein the at least one gene comprises at least one target codon for the incorporation of at least one non-standard amino acid into an expression product of the at least one gene; and
(ii) providing an orthogonal tRNA system comprising a non-standard tRNA and a non-standard aminoacyl-tRNA synthetase (NSAARS), wherein the NSAARS charges the non-standard tRNA with the at least one non-standard amino acid to incorporate the at least one non-standard amino acid into the expression product of the at least one gene.

44. A method according to claim 43, wherein said modifying comprises synthesizing an entire synthetic genome, and introducing the synthetic genome containing the at least one gene encoding an essential protein into the host organism.
